(19) United States
(12) Patent Application Publication
(10) Pub. No.: US 2010/0019191 A1
(43) Pub. Date: Jan. 28, 2010

(54) METHOD FOR PRODUCING 1,2-ETHYLENE GLYCOL AND 1,2-PROPYLENE GLYCOL BY MEANS OF THE HETEROGENEOUSLY CATALYSED HYDROGENOLYSIS OF A POLYOL

(75) Inventors: Bram Willem Hoffer, Heidelberg (DE); Roman Prochazka, Mannheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/518,928

(22) PCT Filed: Dec. 10, 2007
(86) PCT No.: PCT/EP07/63569

§ 371 Date: Jun. 12, 2009

(30) Foreign Application Priority Data
Dec. 15, 2006 (EP) 06126235.8

(51) Int. Cl. C09K 3/18; C07C 31/18

(52) U.S. Cl. 252/70; 568/861

(57) ABSTRACT

A process for preparing 1,2-ethylene glycol and 1,2-propylene glycol by heterogeneously catalyzed hydrogenolysis of a polyol, which comprises using, as a heterogeneous catalyst, a catalyst comprising palladium (Pd) and a support material selected from the group of carbon, zirconium dioxide, titanium dioxide and calcium carbonate, the catalyst not comprising any ruthenium (Ru), and performing the hydrogenolysis in the presence of water.

The present invention relates to a process for preparing 1,2-ethylene glycol and 1,2-propylene glycol by heterogeneously catalyzed hydrogenolysis of a polyol.

1,2-Ethylene glycol (monoethylene glycol, MEG) and 1,2-propylene glycol (propane-1,2-diol) find use as solvents, mineral oil-free lubricants, disinfectants, in brake and hydraulic fluids, as an additive to antifreezes, in organic synthesis, for the production of solvents, polymers, washing raw materials, textile assistants, agents for gas drying.

1,2-Ethylene glycol and 1,2-propylene glycol are prepared generally from the corresponding epoxides (ethylene oxide (EO) and propylene oxide (PO)) (K. Weissermel and H.-J. Arpe, Industrial Organic Chemistry, fourth completely revised version, 2000, WILEY-VCH, Weinheim).

The preparation of 1,2-ethylene glycol and 1,2-propylene glycol by catalytic hydrogenolysis of sugars has been known since the early 1930s.

Usually, the hydrogenolysis reaction is performed at high temperatures (180-260° C.) and high pressures (>200 bar). Highly selective processes for preparing particular glycols are to date unknown.

Both suspension and fixed bed catalysts have been described for the hydrogenolysis process.

Generally employed catalysts comprise Ni, Co or Cu; in the last few years (since about 1980), Ru catalysts have also been described.

U.S. Pat. No. 3,396,199 (Atlas Chem. Ind., Inc.), U.S. Pat. No. 3,030,429 (Inventa AG) and U.S. Pat. No. 4,380,678 (Hydrocarbon Res., Inc.) relate to the use of Ni catalysts in the hydrogenolysis of sugars for the preparation of glycerol.

U.S. Pat. No. 4,401,823 (UOP Inc.) describes the use of particular metal catalysts based on a “shaped carbonaceous pyropolymer”. The preparation of the support is very complicated and the selectivity in the hydrogenolysis is in need of improvement.

U.S. Pat. No. 4,404,411 (Du Pont) teaches the use of hydrogenation catalysts, such as Ni, Pd or Pt catalysts, especially Ni supported on silica/alumina, in the hydrogenolysis of polyols in the presence of bases and of nonaqueous solvents. A disadvantage of organic solvents is that they react in the hydrogenolysis, and expensive solvent is thus lost. A further disadvantage is that organic solvents can form azeotropes with the water of reaction formed and thus cannot be removed completely.

U.S. Pat. No. 4,496,780 (UOP Inc.) relates to the hydrocracking of carbohydrates in the presence of supported noble metal catalysts, especially specific Ru/Ti/Al₂O₃ catalysts.

U.S. Pat. No. 5,026,927 (US department of energy) relates to a homogeneous process for hydrocracking carbohydrates in the presence of soluble transition metal hydrogenation catalysts. The transition metals in the catalytic complexes are especially Ru, Os, Ir, Rh, Fe, Mn, Co. A disadvantage here is the complicated removal of the catalyst from the production mixture after the reaction.

U.S. Pat. No. 5,326,912 (Montecatini Tech. S.r.L.; Novamont S.p.A) describes the use of Ru catalysts which comprise copper and a metal from the group of Pd, Pt, Rh on an activated carbon support for the hydrogenolysis of polyols.

A disadvantage here is the complicated preparation of the catalysts which consist of four components (including support). The substantially complete recovery of the individual expensive noble metals from the catalyst in the course of recycling after its use is also difficult and inconvenient.

EP-A1-510 238 (MENON S.r.I.) relates to the use of sulfide-modified Pt catalysts in the hydrogenolysis of polyols.

C. Montassier et al., “Polyol conversion by liquid phase heterogeneous catalysis over metals”, in Heterogeneous Catalysis and Fine Chemicals, Vol. 41, 1988, pages 165-170 report the use of Cu, Co, Pt, Ru, Ir, Rh/silica catalysts and Co, Ni, Cu Raney catalysts in the hydrogenolysis of sugar alcohols and recommend Cu catalysts.

WO-A-06 092085 (Global Polyol Invest. Ltd.) relates to the preparation of C₂₋₄-dihydroxy alcohols and polyols by hydrocracking sorbitol in the presence of Ni/Ru catalysts.

US-A1-2007/0135301 (Süd-Chemie Inc.) describes, for the hydrogenolysis of carbohydrates, a catalyst comprising nickel on an alumina-silica support.

The present invention was based on the object of discovering an improved, economically viable process for preparing 1,2-ethylene glycol and 1,2-propylene glycol. The chemical conversion of a suitable reactant should afford 1,2-glycols, especially 1,2-ethylene glycol and 1,2-propylene glycol, with high selectivity, yield and space-time yield. Undesired by-products, especially gaseous compounds such as methane, carbon monoxide, carbon dioxide, should be formed in minimum amounts.

Accordingly, a process has been found for preparing 1,2-ethylene glycol and 1,2-propylene glycol by heterogeneously catalyzed hydrogenolysis of a polyol, which comprises using, as a heterogeneous catalyst, a catalyst comprising palladium (Pd) and a support material selected from the group of carbon, zirconium dioxide, titanium dioxide and calcium carbonate, the catalyst not comprising any ruthenium (Ru), and performing the hydrogenolysis in the presence of water.

The polyol used in the process according to the invention is preferably a sugar (also known as a saccharide) or sugar alcohol (=alcohol obtainable from a sugar by reduction), for example a C₃₋₆ sugar or C₃₋₆ sugar alcohol.

Examples of sugars are:

monosaccharides such as glucose, sorbose, tagatose, disaccharides such as maltose, sucrose, lactose, isomaltulose, cellobiose, Palatinose®, oligosaccharides and polysaccharides;
aldotrioses such as glycerylaldehyde, aldotetroses such as erythrose, threose, aldopentoses such as arabinose, ribose, xylose, lyxose, aldohexoses such as allose, altrose, glucose, mannose, gulose, idose, galactose, talose, ketopentoses such as ribulose, ketohexoses such as fructose;
especially D-(+)-glucose.

examples of sugar alcohols are:

erythritol, threitol, Palatinit® (isomalt), arabitol, sorbitol, mannitol, maltitol, lactitol, xylitol;
especially D-sorbitol, D-mannitol, xylitol.

In the process according to the invention, particular preference is given to using sorbitol, glucose or sucrose.

The catalytically active composition of the catalyst is defined as the sum of the masses of the catalytically active constituents and of the support materials, and comprises palladium (Pd) of the 0 oxidation state and/or compounds thereof, for example oxides or hydroxides, and carbon (C), zirconium dioxide (ZrO₂), titanium dioxide (TiO₂) or calcium carbonate (CaCO₃) or mixtures of these support materials, and no ruthenium (Ru) of the 0 oxidation state and/or compounds thereof, for example oxides or hydroxides.

In the process according to the invention, the catalysts are preferably used in the form of catalysts which consist only of catalytically active composition and, if appropriate, a shaping assistant (for example graphite or stearic acid) if the catalyst is used in the form of a shaped body, i.e. do not comprise any further catalytically inactive accompanying substances.

The catalytically active composition can be introduced into the reaction vessel after grinding as powder or as spall, or preferably, after grinding, mixing with shaping assistants, shaping and heat treatment, introduced into the reactor as shaped catalyst bodies—for example as tablets, spheres, rings, extrudates (e.g. strands).

The sum of the abovementioned catalytically active constituents and of the abovementioned support materials in the catalytically active composition—the Pd component being calculated as the metal in the 0 oxidation state—is typically from 80 to 100% by weight, preferably from 90 to 100% by weight, and more preferably from 95 to 100% by weight, especially greater than 99% by weight, for example 100% by weight.

The catalytically active composition of the catalysts used in the process according to the invention comprises in particular from

80 to 99.9% by weight, preferably from 90 to 99.8% by weight, more preferably from 92 to 99.7% by weight, of the support material (carbon, particularly activated carbon, and/or ZrO₂ and/or TiO₂ and/or CaCO₃),
from 0.1 to 20% by weight, preferably from 0.2 to 10% by weight, more preferably from 0.3 to 8% by Weight, of the noble metal Pd, calculated as the metal in the 0 oxidation state, and
from 0 to 20% by weight, preferably from 0 to 10% by weight, more preferably from 0 to 5% by weight, most preferably from 0 to 1% by weight, of one or more elements (oxidation state 0) or the inorganic or organic compounds thereof, selected from groups I A to VI A and I B to VII B of the Periodic Table and the group of Fe, Co, Ni.

Preferred catalysts comprise, in their catalytically active composition, from 80 to 99.9% by weight, preferably from 90 to 99.8% by weight, more preferably from 92 to 99.7% by weight, of carbon, particularly activated carbon, and from 0.1 to 20% by weight, preferably from 0.2 to 10% by weight, more preferably from 0.3 to 8% by weight, of Pd, calculated as the metal in the 0 oxidation state.

Preferred (Ru-free) catalysts do not comprise, in their catalytically active composition, any Ni, Co, Cu, Rh, Pt and/or Ir, preferably any Rh, Pt and/or Ir.

The catalytically active composition of particularly preferred catalysts consists of from 80 to 99.9% by weight, in particular from 90 to 99.8% by weight, more preferably from 92 to 99.7% by weight, of carbon, particularly activated carbon, and from 0.1 to 20% by weight, in particular from 0.2 to 10% by weight, more preferably from 0.3 to 8% by weight, of Pd, calculated as the metal in the 0 oxidation state.

For the carbon support material, preference is given to carbon black, graphite and especially activated carbon.

The catalysts used in the process according to the invention have, in the case of activated carbon as the support material, a surface area (to DIN 66131) of preferably from 500 to 2000 m²/g, more preferably from 500 to 1800 m²/g, and a pore volume (to DIN 66134) of preferably from 0.05 to 5.0 cm³/g, more preferably from 0.10 to 3 cm³/g.

For the preparation of the catalysts used in the process according to the invention, various processes are possible.

The catalysts used in the process according to the invention are preferably prepared by impregnating carbon, especially activated carbon, zirconium dioxide (ZrO₂), titanium dioxide (TiO₂), calcium carbonate (CaCO₃) or mixtures of two or more of these support materials, which are present, for example, in the form of powder, spall or shaped bodies such as extrudates, tablets, spheres or rings.

Zirconium dioxide is used for catalyst preparation, for example, in the monoclinic or tetragonal form, preferably in the monoclinic form, and titanium dioxide, for example, as anatase or rutile.

Activated carbon is used with a surface area (to DIN 66131) of preferably from 500 to 2000 m²/g, more preferably from 500 to 1800 m²/g, and a pore volume (to DIN 66134) of preferably from 0.05 to 5.0 cm³/g, more preferably from 0.10 to 3 cm³/g.

Examples of such activated carbons are the commercially available Norit® SX types from Norit (The Netherlands).

According to whether the catalyst is to be prepared as a suspension catalyst or fixed bed catalyst, the carbon support material is used in pulverulent form or in the form of extrudates, spheres, spall, etc. Before it is doped, the carbon support can be pretreated, for instance by oxidation with nitric acid, oxygen, hydrogen peroxide, hydrochloric acid, etc.

Shaped bodies of the abovementioned support materials can be prepared by the customary processes.

These support materials are likewise impregnated by the customary processes, as described, for example, in EP-A-599 180, EP-A-673 918 or A. B. Stiles, Catalyst Manufacture—Laboratory and Commercial Preparations, Marcel Dekker, pages 89 to 91, New York (1983), by applying a metal salt solution appropriate in each case in one or more impregnation stages, the metal salts used being, for example, appropriate nitrates, acetates or chlorides, After the impregnation, the composition is dried and if appropriate calcined.

The impregnation can be effected by the so-called incipient wetness method, in which the oxidic support material is moistened up to no further than saturation with the impregnation solution according to its water uptake capacity. However, the impregnation can also be effected in supernatant solution.

In multistage impregnation processes, it is appropriate to dry and, if appropriate, to calcine between individual impregnation steps. Multistage impregnation is advantageously employable especially when the support material is to be contacted with a relatively large amount of metal.

For the application of a plurality of metal components to the support material, the impregnation can be effected simultaneously with all metal salts or in any sequence of the individual metal salts in succession.

A special case of impregnation is that of spray-drying, in which the catalyst support mentioned is sprayed in a spray dryer with the component(s) to be applied in a suitable solvent. What is advantageous in this variant is the combination of application and drying of the active component(s) in one step.

The catalyst can also be prepared by precipitating the metal salts on the support, as described, for example, in EP-A-1 317 959 (BASF AG).

The catalysts prepared in this way comprise the catalytically active metal(s) such as Pd in the form of a mixture of its/their oxygen compound(s), i.e. especially as oxides and mixed oxides.

The catalysts used in the process according to the invention may be reduced before use. The reduction can be effected at ambient pressure or under pressure. When reduction is effected under ambient pressure, the procedure is to heat the catalyst under inert gas, for example nitrogen, up to the reduction temperature and then gradually to replace the inert gas with hydrogen.

In a reduction under pressure, the procedure is conveniently to also undertake the reduction at the pressures and temperatures employed later in the process according to the invention. The duration of the reduction is selected according to temperature and hydrogen pressure, i.e. the more severe the conditions, the shorter the reduction time selected can be.

In general, reduction is effected at a temperature of from 80 to 250° C., a hydrogen pressure of from 0.5 to 350 bar and a duration of from 1 to 48 h.

However, it is likewise possible to use the unreduced catalysts in the process according to the invention. In this case, the reduction of the particular catalyst is then effected simultaneously under the process conditions. After a short operating time of the process according to the invention of a few hours or a few days, the reduction of the catalyst is typically virtually complete.

Examples of catalysts usable in the process according to the invention are supported catalysts according to WO-A-96/36589 (BASF AG), which comprise palladium and, as a support material, activated carbon, titanium dioxide and/or zirconium dioxide.

Further examples of catalysts usable in accordance with the invention are the following commercially available catalysts:

5% by weight of Pd on calcium carbonate, 5% by weight of Pd on carbon powder, “The catalyst technical handbook” (2005) Johnson Matthey PLC company brochure, page 69, and
5% by weight of Pd on carbon, “Edelmetall-Katalysatoren” W.C. Heraeus company brochure (2003), page 18.

In the process according to the invention, the polyol (reactant) is hydrogenolyzed in the liquid phase, i.e. dissolved or suspended in water and optionally a further solvent or diluent.

Useful additional solvents or diluents as well as the water are in particular those which are capable of dissolving the reactant substantially completely or mix completely with it and are inert under the process conditions.

Examples of additional suitable solvents and diluents as well as water are:

aliphatic alcohols, especially C₁₋₈-alcohols, particularly C₁₋₄-alcohols, such as methanol, ethanol, n- or isopropanol, n-, 2-, iso- or tert-butanol.

Particular preference is given to performing the hydrogenolysis in the presence of water as the sole solvent or diluent.

The concentration of reactant (polyol) in the liquid phase is preferably in the range from 10 to 80% by weight, more preferably from 30 to 70% by weight, based in each case on the total weight of the solution or suspension (without catalyst).

A further increase in the selectivity can be achieved by adding a basic compound (a base), as described, for example, in U.S. Pat. No. 5,107,018 (BASF AG) or U.S. Pat. No. 4,404,411 (see above).

Examples of advantageous bases are alkali metal hydroxides, alkaline earth metal hydroxides, alkali metal carbonates, alkaline earth metal carbonates, alkali metal oxides, alkaline earth metal oxides, alkali metal alkoxides, alkaline earth metal alkoxides and nitrogen bases, for example tetraalkylammonium hydroxides or carbonates.

Preference is given to oxides, hydroxides and carbonates of alkaline earth metals, especially alkali metals, and combinations thereof.

Particular preference is given to lithium hydroxide (LiOH), calcium oxide (CaO), sodium hydroxide (NaOH), magnesium hydroxide (Mg(OH)₂) and sodium carbonate (Na₂CO₃).

Preference is given to performing the hydrogenolysis in the presence of from 0.01 to 30% by weight, particularly from 0.1 to 25% by weight, more particularly from 0.5 to 5% by weight, of the basic compound, based in each case on the polyol used.

The process can be performed continuously, discontinuously or semicontinuously. Preference is given to a continuous method.

Preferred reactors are tubular reactors with a fixed catalyst bed for the continuous method, autoclaves or bubble columns for the discontinuous method and autoclaves for the semicontinuous method.

The hydrogenolysis is performed preferably at a temperature in the range from 150 to 325° C., more preferably from 175 to 300° C., in particular from 200 to 275° C.

The hydrogenolysis is preferably performed at an absolute pressure in the range from 50 to 325 bar, more preferably from 75 to 300 bar, in particular from 100 to 275 bar.

In the batchwise hydrogenolysis, the reactant (polyol) is initially charged in the reactor as a solution or suspension, preferably as a solution, in water and, if appropriate, a further suitable solvent or diluent (see above); the catalyst material is suspended in the reactant and solvent or diluent (see above). In order to ensure a high conversion and high selectivity, the solution or suspension, catalyst and hydrogen gas have to be mixed well, for example by a turbine stirrer in an autoclave. The suspended catalyst material can be introduced and removed again with the aid of common techniques (sedimentation, centrifugation, cake filtration, crossflow filtration). The catalyst can be used once or more than once. The catalyst concentration is advantageously from 0.1 to 20% by weight, preferably from 0.5 to 10% by weight, in particular from 1 to 5% by weight, based in each case on the total weight of the solution or suspension (total weight with catalyst). The mean particle size of the catalyst is advantageously in the range from 0.001 to 1 mm, preferably in the range from 0.005 to 0.5 mm, in particular from 0.01 to 0.25 mm.

In the continuous hydrogenolysis, the reactant (polyol) is passed as a solution or suspension, preferably as a solution, in water and, if appropriate, a further suitable solvent or diluent (see above), in the liquid phase including hydrogen, over the catalyst which is preferably disposed in a (preferably externally) heated fixed bed reactor. Both a trickle mode and a liquid phase mode are possible. The catalyst loading is generally in the range from 0.05 to 5 kg, preferably from 0.1 to 2 kg, more preferably from 0.2 to 1 kg, of polyol per liter of catalyst (bed volume) and hour. It is appropriate to heat the reactant solution or reactant suspension actually before the feeding into the reaction vessel, and preferably to the reaction temperature.

It is also possible to employ a continuous suspension mode, as described, for example, in EP-A2-1 318 128 (BASF AG) or in FR-A-2 603 276 (Inst. Français du Pétrole).

The pressure in the reaction vessel, which arises from the sum of the partial pressures of the reactant, of the reaction products formed and of the solvent or diluent at the particular temperature, is appropriately increased to the desired reaction pressure by injecting hydrogen.

On completion of hydrogenolysis, the 1,2-ethylene glycol and 1,2-propylene glycol products can be removed again from the water and from any solvent or diluent used. This is done by processes known to those skilled in the art, for example by distilling off the solvent(s) or diluent(s), optionally under reduced pressure.

Alternative separation processes are described, for example, in U.S. Pat. No. 6,548,681 (Michigan State Univ.) and Dhale et al. Chem. Eng. Sci, 59 (2004), pages 2881-2890.

Unconverted reactants and any suitable by-products which occur can be recycled back into the synthesis.

According to the prior art, 1,2-ethylene glycol- and 1,2-propylene glycol-based antifreezes are produced by synthesizing these glycols starting from naphtha or the like over several stages [ethylene → ethylene oxide (EO) → monoethylene glycol (MEG); propylene → propylene oxide (PO) → monopropylene glycol (MPG)] in a complicated manner separately by means of several plants and then mixing them.

The process according to the invention affords, after workup of the crude process product, a 1,2-ethylene glycol-1,2-propylene glycol mixture which can be used advantageously and inexpensively for the production of an antifreeze.

The invention thus also provides a process for producing an antifreeze, which comprises preparing a mixture of 1,2-ethylene glycol and 1,2-propylene glycol by the inventive hydrogenolysis process as described above, admixing the mixture with water and optionally adding a corrosion protection component.

The 1,2-ethylene glycol-1,2-propylene glycol mixture consists preferably of from 20 to 90% by weight of ethylene glycol and from 10 to 80% by weight of propylene glycol.

For the production of the antifreeze, the glycol mixture is admixed preferably with from 40 to 90% by weight of water, based on the weight of the glycol mixture.

The amount of corrosion protection component which is selected, for example, from the group of carboxylic acids, molybdates or triazoles is, for example, in the range from 1 to 10% by weight, based on the total weight of the antifreeze.

The antifreeze is used especially advantageously for coolant circuits of internal combustion engines; see, for example, WO-A1-06/092376 (BASF AG).

### EXAMPLES

### Gas Chromatography Methods

Gas sample: separation phases/methods

| Column | Length (m) | Diameter (mm) | Components |
|---|---|---|---|
| Molecular sieve | 25 | 0.53 | H₂, He |
| Poraplot Q | 40 | 0.53 | CO2, hydrocarbons |
| | | | (≧1% by vol.) |
| 5 Å molecular sieve | 25 | 0.53 | Inerts, methane |
| HP-Alu | 50 | 0.32 | Hydrocarbons |
| | | | (<1% by vol.) |

Liquid Sample:

Separation column: RTX-5 Amine (30 m×0.32 mm) with He as the carrier gas.
Temperature program: 50° C. (hold for 15 min.), heat to 280° C. at 10° C./min, hold for 30 min. The injector temperature is 280° C., the FID detector temperature 300° C.

### 1. Preparation of Lower Polyhydric Alcohols, Especially 1,2-ethylene glycol and 1,2-propylene glycol

A Pd/C catalyst comprising 5% by weight of Pd was used to convert sorbitol to lower polyhydric alcohols by the following method. 150 g of an aqueous solution comprising 15 g of sorbitol, 750 mg of calcium oxide and 7.5 g of catalyst are introduced into an autoclave having a capacity of 300 cm³. The autoclave is sealed, and the air present therein is then driven out by purging with nitrogen. The inert gas is then replaced with hydrogen and the autoclave is injected with hydrogen at ambient temperature up to a pressure of 5.0 MPa. The autoclave is then heated and stirred at 1000 rpm. After about 30 minutes, a temperature of 230° C. is attained and is retained for 10 hours. After about 1 hour, the pressure is increased to about 25.0 MPa by introducing hydrogen. The pressure is kept at 25.0 MPa by constantly introducing fresh hydrogen. After the 10 hours have expired, the autoclave is cooled to ambient temperature, and a gas sample is then taken for an analysis before the autoclave is decompressed. The reaction liquid is then separated from the catalyst by filtration. The gas sample is analyzed by means of gas chromatography in order to demonstrate the presence of hydrocarbons (methane, ethane, ethylene, etc.) and carbon dioxide. The reaction liquid is analyzed by means of gas chromatography. It comprises mainly ethanediol, 1,2-propylene glycol, ethanol and 1-propanol, and smaller amounts of glycerol and butanediol. The sorbitol conversion was 100%.

### Comparative Examples

### 2. Preparation of Lower Polyhydric Alcohols, Especially 1,2-ethylene glycol and 1,2-propylene glycol

A Pd/γ-Al₂O₃ catalyst comprising 4.7% by weight of Pd was used to convert sorbitol to lower polyhydric alcohols by the following method. 150 g of an aqueous solution comprising 15 g of sorbitol, 750 mg of calcium oxide and 8 g of catalyst are introduced into an autoclave having a capacity of 300 cm³. The autoclave is sealed, and the air present therein is then driven out by purging with nitrogen. The inert gas is then replaced with hydrogen and the autoclave is injected with hydrogen at ambient temperature up to a pressure of 5.0 MPa. The autoclave is then heated and stirred at 1000 rpm. After about 30 minutes, a temperature of 230° C. is attained and is retained for 16 hours. After about 1 hour, the pressure is increased to about 25.0 MPa by introducing hydrogen. The pressure is kept at 25.0 MPa by constantly introducing fresh hydrogen. After the 16 hours have expired, the autoclave is cooled to ambient temperature, and a gas sample is then taken for an analysis before the autoclave is decompressed. The reaction liquid is then separated from the catalyst by filtration. The reaction liquid is analyzed by means of gas chromatography. It comprises mainly ethanediol, 1,2-propylene glycol, ethanol and 1-propanol, and smaller amounts of glycerol and butanediol. The sorbitol conversion was only 88%.

### 3. Preparation of Lower Polyhydric Alcohols

An Ru/C catalyst comprising 5% by weight of Ru was used to convert sorbitol to lower polyhydric alcohols by the following method. 150 g of an aqueous solution comprising 15 g of sorbitol, 750 mg of calcium oxide and 7.5 g of catalyst are introduced into an autoclave having a capacity of 300 cm³. The autoclave is sealed, and the air present therein is then driven out by purging with nitrogen. The inert gas is then replaced with hydrogen and the autoclave is injected with hydrogen at ambient temperature up to a pressure of 5.0 MPa. The autoclave is then heated and stirred at 1000 rpm. After about 30 minutes, a temperature of 230° C. is attained and is retained for 10 hours. After about 1 hour, the pressure is increased to about 25.0 MPa by introducing hydrogen. The pressure is kept at 25.0 MPa by constantly introducing fresh hydrogen. After the 10 hours have expired, the autoclave is cooled to ambient temperature, and a gas sample is then taken for an analysis before the autoclave is decompressed. The reaction liquid is then separated from the catalyst by filtration. The gas sample is analyzed by means of gas chromatography in order to demonstrate the presence of hydrocarbons (methane, ethane, ethylene, etc.) and carbon dioxide. The reaction liquid is analyzed by means of gas chromatography. It comprises mainly ethanediol, 1,2-propylene glycol, ethanol and 1-propanol, and smaller amounts of glycerol and butanediol. The sorbitol conversion was 100%.

### 4. Preparation of Lower Polyhydric Alcohols

A catalyst described in U.S. Pat. No. 5,210,335 (BASE AG)(72% Co, 21% Cu, 7% Mn) was used to convert sorbitol to lower polyhydric alcohols by the following method. 175 g of an aqueous solution comprising 37.5 g of sorbitol, 2.5 g of sodium hydroxide and 10 g of catalyst are introduced into an autoclave having a capacity of 270 cm³. The autoclave is sealed, and the air present therein is then driven out by purging with nitrogen. The inert gas is then replaced with hydrogen and the autoclave is injected with hydrogen at ambient temperature up to a pressure of 15.0 MPa. The autoclave is then heated and stirred at 1000 rpm. After about 40 minutes, a temperature of 230° C. is attained and is retained for 3 hours. After about 3 hours, the pressure is increased to about 25.0 MPa by introducing hydrogen and the temperature is increased to 250° C. The pressure is kept at 25.0 MPa by continuously introducing fresh hydrogen. After a further 10 hours, the autoclave is cooled to ambient temperature, and a gas sample is then taken for an analysis before the autoclave is decompressed. The reaction liquid is then separated from the catalyst by filtration. The gas sample is analyzed by means of gas chromatography in order to demonstrate the presence of hydrocarbons (methane, ethane, ethylene, etc.) and carbon dioxide. The reaction liquid is analyzed by means of gas chromatography. It comprises mainly ethanediol, 1,2-propylene glycol, ethanol and 1-propanol, and smaller amounts of glycerol and butanediol. The sorbitol conversion was 100%.

TABLE 1
Liquid phase composition (% peak area)

| | Sorbitol | Ethanediol | 1,2-Propylene glycol | Others |
|---|---|---|---|---|
| Example 1 | 0 | 21.0 | 48.9 | 30.1 |
| Example 2 | 12 | 16.4 | 48.2 | 35.4 |
| Example 3 | 0 | 5.5 | 66.8 | 27.7 |
| Example 4 | 0 | 13.4 | 38.5 | 51.9 |

TABLE 2
Gas phase composition (% by vol.)

| | Methane | Ethane | Nitrogen | CO₂ | H₂ |
|---|---|---|---|---|---|
| Example 1 | 0.92 | 0.01 | — | <0.03 | Remainder |
| Example 2 | — | — | — | — | — |
| Example 3 | 59.9 | 2.12 | — | 3.62 | 5.27 |
| Example 4 | 5.8 | 0.16 | 1.7 | <0.03 | Remainder |

“—” means: not measured

The catalyst 1 used in accordance with the invention produces 1,2-ethylene glycol (=ethanediol) and 1,2-propylene glycol selectively without significant methanization taking place; in this regard, see, in contrast, the catalysts from Examples 3 and 4.

In Example 2, only 88% sorbitol conversion is achieved after 16 hours. The catalyst 1 used in accordance with the invention exhibited full conversion even after 10 hours, i.e. is much more active.

### Further Inventive Example

A Pd/C catalyst comprising 5% by weight of Pd was used to convert sorbitol to lower polyhydric alcohols by the following method. 150 g of an aqueous solution comprising 15 g of sorbitol, 750 mg of calcium oxide and 7.5 g of catalyst are introduced into an autoclave having a capacity of 270 cm³. The autoclave is sealed, and the air present therein is then driven out by purging with nitrogen. The inert gas is then replaced with hydrogen and the autoclave is injected with hydrogen at ambient temperature up to a pressure of 5.0 MPa. The autoclave is then heated and stirred at 1000 rpm. After about 1 hour, a temperature of 230° C. is attained and is retained for 8 hours. After about 1 hour, the pressure is increased to about 25.0 MPa by introducing hydrogen. The pressure is kept at 25.0 MPa by constantly introducing fresh hydrogen. During the experiment, samples are taken from the reaction liquid. The reaction liquid is separated from the catalyst by filtration and analyzed by means of gas chromatography. It comprises mainly ethanediol, 1,2-propylene glycol, ethanol and 1-propanol, and smaller amounts of glycerol and butanediol. The time-resolved liquid phase compositions are listed in Table 3.

TABLE 3
Liquid phase composition (% peak area)

| | | | | 1,2-Propylene | |
|---|---|---|---|---|---|
| | Sorbitol | Glycerol | Ethanediol | glycol | Others |
| Start | 97.1 | 0 | 0 | 0 | 2.9 |
| 2 h | 39.4 | 7.0 | 11.1 | 27.8 | 14.7 |
| 4 h | 2.9 | 14.7 | 20.6 | 48.0 | 13.8 |
| 6 h | 1.1 | 12.8 | 20.5 | 49.8 | 15.8 |
| 8 h | 0 | 8.4 | 21.5 | 55.0 | 15.1 |
| 10 h | 0 | 5.5 | 21.2 | 57.2 | 16.1 |
| After the | 0 | 3.2 | 22.0 | 60.1 | 14.7 |
| experiment | | | | | |

1-15. (canceled)

16. A process for preparing 1,2-ethylene glycol and 1,2-propylene glycol by heterogeneously catalyzed hydrogenolysis of a polyol, which comprises using, as a heterogeneous catalyst, a catalyst comprising palladium (Pd) and a support material selected from the group of carbon, zirconium dioxide, titanium dioxide and calcium carbonate, the catalyst not comprising any ruthenium (Ru), and performing the hydrogenolysis in the presence of water.

17. The process according to claim 16, wherein the polyol is a sugar or sugar alcohol.

18. The process according to claim 17, wherein the sugar is glucose, fructose, sorbose, tagatose, mannose, galactose, arabinose, xylose, lyxose, ribose, sucrose, lactose, maltose, isomaltose or cellobiose.

19. The process according to claim 17, wherein the sugar alcohol is sorbitol, mannitol, erythritol, threitol, isomalt, arabitol, lactitol, maltitol or xylitol.

20. The process according to claim 16, wherein the catalytically active composition of the heterogeneous catalyst comprises from 80 to 99.9% by weight of the support material and from 0.1 to 20% by weight of Pd, calculated as the metal in the 0 oxidation state.

21. The process according to claim 16, wherein the catalytically active composition of the heterogeneous catalyst comprises from 90 to 99.8% by weight of activated carbon and from 0.2 to 10% by weight of Pd, calculated as the metal in the 0 oxidation state.

22. The process according to claim 16, wherein the catalytically active composition of the heterogeneous catalyst does not comprise any Rh, Pt or Ir.

23. The process according to claim 16, wherein the hydrogenolysis is performed in the presence of a basic compound and said basic compound is an alkali metal hydroxide, an alkaline earth metal hydroxide, an alkali metal oxide, an alkaline earth metal oxide, an alkali metal carbonate or an alkaline earth metal carbonate.

24. The process according to claim 23, wherein the hydrogenolysis is performed in the presence of from 0.1 to 25% by weight of the basic compound based on the polyol used.

25. The process according to claim 23, wherein the basic compound used is CaO or NaOH.

26. The process according to claim 16, wherein the hydrogenolysis is performed at a temperature in the range from 150 to 325° C.

27. The process according to claim 16, wherein the hydrogenolysis is performed at an absolute pressure in the range from 50 to 325 bar.

28. The process according to claim 16, wherein the hydrogenolysis is performed in the presence of water as a solvent or diluent, the concentration of polyol in the liquid phase being in the range from 10 to 80% by weight based on the total weight of the solution or suspension (without catalyst).

29. The process according to claim 16, wherein the hydrogenolysis is performed in fixed bed or suspension mode.

30. A process for producing an antifreeze, which comprises preparing a mixture of 1,2-ethylene glycol and 1,2-propylene glycol by a hydrogenolysis process according to claim 16, admixing the mixture with water and optionally adding a corrosion protection component.
